(19) United States
(12) Patent Application Publication
(10) Pub. No.: US 2013/0190557 A1
(43) Pub. Date: Jul. 25, 2013

(54) GASTRIC BANDS FOR REDUCING OBSTRUCTIONS

(71) Applicants: Jason Hoover, Goleta, CA (US); Zachary P. Dominguez, Santa Barbara, CA (US); Justin Schwab, Santa Barbara, CA (US); Babak Honaryar, Orinda, CA (US)

(72) Inventors: Jason Hoover, Goleta, CA (US); Zachary P. Dominguez, Santa Barbara, CA (US); Justin Schwab, Santa Barbara, CA (US); Babak Honaryar, Orinda, CA (US)

(73) Assignee: ALLERGAN, INC., Irvine, CA (US)

(21) Appl. No.: 13/356,459

(22) Filed: Jan. 23, 2012

(51) Int. Cl. A61F 2/04

(52) U.S. Cl. 600/37

(57) ABSTRACT

Generally described herein are apparatus, systems and methods related to gastric bands which provide increased compliance to reduce food obstructions and/or reduces over restriction causing symptoms such as gastric enlargement and pouch dilatation. In one embodiment, a dual ringed reservoir band or inflatable portion is provided. In one embodiment, an additional ring or a middle pouch may be added to the dual ringed reservoir band. The addition of an additional ring or middle pouch may further increase band compliance resulting in even fewer food obstructions. In another embodiment, one or more funnels can also be implemented into a gastric banding system to induce satiety and/or for guiding a bolus through the gastric band.

### FIELD

The present invention generally relates to medical systems, devices and uses thereof for treating obesity and/or obesity-related diseases. More specifically, the present invention relates to gastric bands for reducing the occurrence of an obstruction caused by a bolus of food in the esophageal gastric junction.

### BACKGROUND

Gastric banding apparatus have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND APO (Allergan, Inc., Irvine, Calif.) gastric band. Generally, gastric bands are placed about the cardia, or upper portion, of a patient's stomach forming a stoma that restricts the food's passage into a lower portion of the stomach. When the stoma is of an appropriate size that is restricted by a gastric band, food held in the upper portion of the stomach may provide a feeling of satiety or fullness that discourages overeating. Unlike gastric bypass procedures, gastric band apparatus are reversible and require no permanent modification to the gastrointestinal tract. An example of a gastric banding system is disclosed in Roslin, et al., U.S. Patent Pub. No. 2006/0235448, the entire disclosure of which is incorporated herein by this specific reference.

However, a large bolus of food swallowed by a patient may temporarily cause an obstruction leading the patient to possibly experience discomfort. Accordingly, some attempts have been made to provide an alternatively configured gastric band.

For example, Gilbert, FR2922097, discloses a dual-reservoir gastric band as illustrated in FIG. 1A. However, the gastric band of Gilbert remains lacking as the fluid transfer region between the reservoirs is not virtually 360° and might not provide increased compliance. Instead, the gastric band of Gilbert appears to require a certain minimum threshold of pressure to operate a portion of the fluid transfer region.

In another example, the “Soft Basket Band” as illustrated in FIG. 1B illustrates a basket attached to the band for preventing dilatation of the esophageal-gastric junction. However, the Soft Basket Band of FIG. 1B suffers from the drawback that the spacing in the basket might not be optimal and may allow tissue to extrude itself, and further the basket might not be supportive enough due to the number and sizing of the spacing. In addition, the basket itself is not funnel-shaped.

What is needed is a gastric band of increased compliance which reduces the occurrence of food obstructions and/or reduces over restriction causing symptoms such as gastric enlargement and pouch dilatation.

### SUMMARY

Generally described herein are apparatus, systems and methods related to gastric bands which provide increased compliance to reduce food obstructions and/or reduce over restriction causing symptoms such as gastric enlargement and pouch dilatation.

In one embodiment, a dual ringed reservoir band or inflatable portion is provided. As the bolus applies pressure to the top reservoir or ring, fluid is temporarily transferred to the bottom reservoir or ring thereby allowing the bolus to move downward along the patient's esophageal junction. As the bolus reaches the bottom reservoir or ring, fluid is transferred back up to the top reservoir or ring to allow the bolus to pass the dual ringed reservoir band or inflatable portion and further move down the patient's digestive tract.

In one embodiment, an additional ring or a middle pouch may be added to the dual ringed reservoir band. The addition of an additional ring or middle pouch may further increase band compliance resulting in even fewer food obstructions.

In one embodiment, one or more funnels can also be implemented into a gastric banding system for guiding a bolus through the gastric band. As a result, improved use of the green zone, which may be the optimal zone related to gastric banding adjustment that provides early and prolonged satiety and/or satisfactory weight loss or maintenance may be achieved. Furthermore, the funnel may reduce the number of food obstructions, as well as increase the variety of foods allowed to be eaten by the patient, by providing a smooth, streamlined transition. The funnel shape also prevents the formation of an inadvertent esophageal dilatation and pouch formation just above the gastric band. This undesired pouch or dilatation can result in dormant/residual food in the pouch which will eventually decay and may even result in a surgical explantation of the gastric band. The funnel geometry supports the esophageal tissue just above the gastric band and prevents the formation of the pouch. In addition, the funnel geometry at the other end of the gastric band which faces the stomach provides a more conformal fit with the geometry of the larger stomach and can prevent slippage of the gastric band.

In one embodiment, an inflatable portion apparatus for use within a gastric banding system for the treatment of obesity, the inflatable portion apparatus including a first adjustably filled reservoir having a first surface configured to contact and form a constriction about a first portion of a patient's esophageal-gastric junction, a second adjustably filled reservoir in fluid communication with the first reservoir and having a second surface configured to contact and form a constriction about a second portion of a patient's esophageal-gastric junction, and a bi-directional fluid transfer component positioned between the first adjustably filled fluid reservoir and the second adjustably filled fluid reservoir for improving the compliance of the first adjustably filled reservoir and the second adjustably filled reservoir, the bi-directional fluid transfer component configured to transfer fluid from the first adjustably filled reservoir to the second adjustably filled fluid reservoir in response to a bolus exerting a pressure on the first surface of the first adjustably filled reservoir, and further configured to transfer fluid from the second adjustably filled reservoir to the first adjustably filled fluid reservoir in response to the bolus exerting a pressure on the second surface of the second adjustably filled reservoir.

In one embodiment, a gastric banding device comprising an inflatable portion for use for the treatment of obesity, the inflatable portion including a first adjustably filled reservoir configured to displace fluid in response to a bolus causing pressure on the first adjustably filled reservoir, a second adjustably filled reservoir fluidly separated from the first adjustably filled reservoir, and configured to receive fluid displaced from the first adjustably filled reservoir, and further configured to displace fluid in response to the bolus causing pressure on the second adjustably filled reservoir, and a first valve for allowing fluid communication between the first adjustably filled reservoir and the second adjustably filled reservoir.

In one embodiment, a dual-reservoir, dual-funnel gastric banding device usable for treatment of obesity, comprising a first reservoir forming a top section of a funnel portion, the first reservoir adjustably filled with fluid, a second reservoir fluidly coupled to the first reservoir forming a bottom section of an inverted funnel portion, the second reservoir adjustably filled with fluid, a middle pouch forming a bottom section of the funnel portion, and a top section of the inverted funnel portion, the middle pouch adjustably filled with fluid and fluidly coupled to the first reservoir and the second reservoir, a first fluid transfer component forming a middle section of the funnel portion, and for transferring fluid between the first reservoir and the middle pouch, and a second fluid transfer component forming a middle section of the inverted funnel portion, and for transferring fluid between the middle pouch and the second reservoir.

In one embodiment, a single-reservoir, single-funnel gastric banding device usable for treatment of obesity, comprising a first reservoir forming a top section of a funnel portion configured to guide a bolus swallowed by a patient, the first reservoir adjustably filled with fluid, a second reservoir fluidly coupled to the first reservoir forming a bottom section of the funnel portion, the second reservoir adjustably filled with fluid, a fluid transfer component forming a middle section of the funnel portion, and for transferring fluid between the first reservoir and the second reservoir.

In one embodiment, a gastric banding system for the treatment of obesity, the gastric banding system comprising an inflatable portion adjustably filled with fluid and configured to provide constriction on an esophageal gastric junction of a patient, a ring coupled to an outside surface of the inflatable portion configured to provided structural support, a funnel portion integrated with an inside surface of the inflatable portion, the funnel portion configured to guide a bolus swallowed by the patient, an access port coupled to the inflatable portion for the addition and removal of fluid from the inflatable portion, and a tube for fluidly connecting the inflatable portion and the access port.

### BRIEF DESCRIPTION OF THE DRAWINGS

The features, obstacles, and advantages of the present invention will become more apparent from the detailed description set forth below when taken in conjunction with the drawings, wherein:

FIG. 1A illustrates a prior art gastric band.

FIG. 1B illustrates another prior art gastric band.

FIG. 2A illustrates a gastric banding system according to one or more embodiments of the present invention.

FIG. 2B illustrates the gastric banding system of FIG. 2A shown outside the patient's body according to one or more embodiments of the present invention.

FIG. 3 illustrates a dual-ring reservoir band according to one or more embodiments of the present invention.

FIG. 4A illustrates the behavior of the dual-ring reservoir band of FIG. 3 when a bolus of food is at a top ring location according to one or more embodiments of the present invention.

FIG. 4B illustrates the behavior of the dual-ring reservoir band of FIG. 3 when a bolus of food is between the top ring location and the bottom ring location according to one or more embodiments of the present invention.

FIG. 4C illustrates the behavior of the dual-ring reservoir band of FIG. 3 when the bolus of food is at the bottom ring location according to one or more embodiments of the present invention.

FIG. 5A illustrates a dual-ring reservoir band having flush contact points according to one or more embodiments of the present invention.

FIG. 5B illustrates a dual-ring reservoir with rectangular cross section, that incorporates two communicating fluid chambers, according to one or more embodiments of the present invention.

FIG. 5C illustrates a dual-ring reservoir having various wall thicknesses associated with the fluid compartments, which may provide a comparatively restrictive top ring and a comparatively compliant bottom ring according to one or more embodiments of the present invention.

FIG. 5D illustrates a dual-ring reservoir having a thin wall thickness, which may affect rate of fluid communication, and compliance of the implant against the anatomy according to one or more embodiments of the present invention.

FIG. 6 illustrates a dual-ring reservoir having a valve system for transferring fluid from the top reservoir to the bottom reservoir and vice versa according to one or more embodiments of the present invention.

FIG. 7 illustrates a cross-sectional view of a dual-ended funnel-shaped band positioned about a patient's gastro-esophageal junction according to one or more embodiments of the present invention.

FIG. 8A illustrates a dual-ended funnel-shaped band according to one or more embodiments of the present invention.

FIG. 8B illustrates the behavior of the dual-ended funnel-shaped band of FIG. 8A when a bolus of food is passing through the dual-ended funnel-shaped band of FIG. 8A according to one or more embodiments of the present invention.

FIG. 8C illustrates the behavior of the dual-ended funnel-shaped band of FIG. 8A when a bolus of food reaches the center of the dual-ended funnel-shaped band of FIG. 8A according to one or more embodiments of the present invention.

FIG. 9 illustrates a dual-ended funnel-shaped band according to one or more embodiments of the present invention.

FIG. 10A illustrates a single-ended funnel-shaped band according to one or more embodiments of the present invention.

FIG. 10B illustrates the behavior of the single-ended funnel-shaped band of FIG. 10A when a bolus of food is passing through the single-ended funnel-shaped band of FIG. 10A according to one or more embodiments of the present invention.

FIG. 10C illustrates the behavior of the dual-ended funnel-shaped band of FIG. 10A when a bolus of food reaches the middle fluid pouch of the single-ended funnel-shaped band of FIG. 10A according to one or more embodiments of the present invention.

FIG. 11A illustrates a dual-ended funnel-shaped band according to one or more embodiments of the present invention.

FIG. 11B illustrates a cross sectional view of the dual-ended funnel-shaped band of FIG. 11A according to one or more embodiments of the present invention.

FIG. 12A illustrates a single-ended funnel-shaped band according to one or more embodiments of the present invention.

FIG. 12B illustrates a cross sectional view of the single-ended funnel-shaped band of FIG. 12A according to one or more embodiments of the present invention.

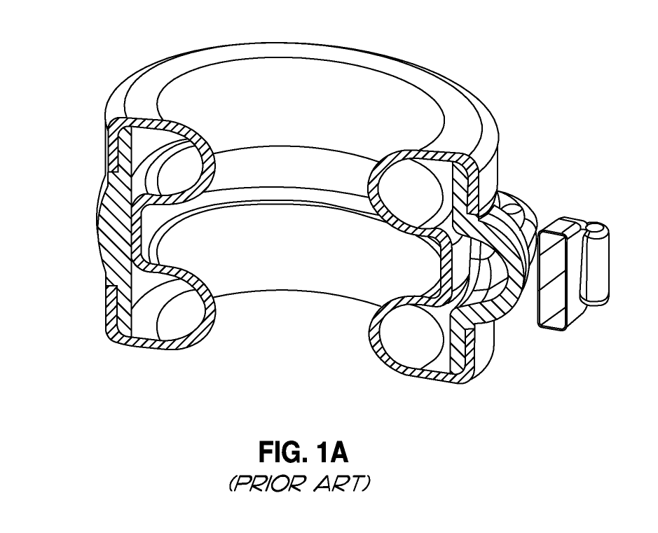

### DETAILED DESCRIPTION

Apparatuses, systems and/or methods that implement the embodiments of the various features of the present invention will now be described with reference to the drawings. The drawings and the associated descriptions are provided to illustrate some embodiments of the present invention and not to limit the scope of the present invention. Throughout the drawings, reference numbers are re-used to indicate correspondence between referenced elements.

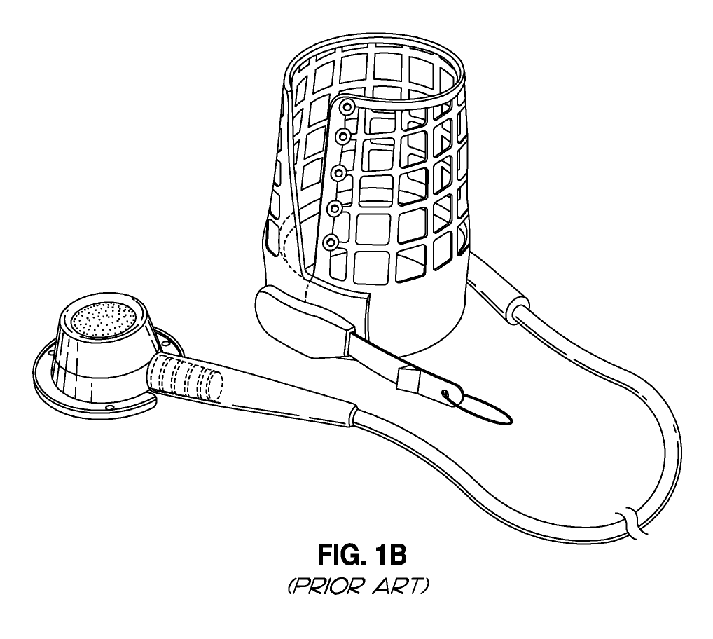

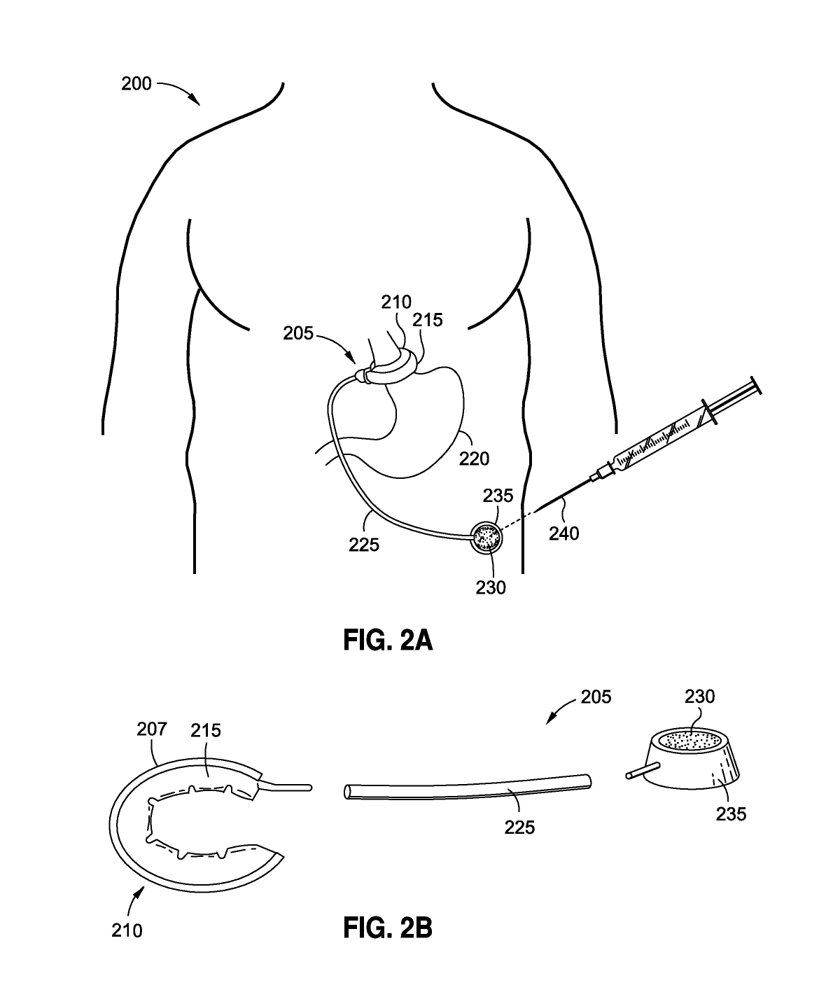

FIG. 2A illustrates an implantable gastric banding system 205 used for the treatment of obesity. In the embodiment shown, a tube 225 (or a catheter) and an access port 230 are used in the implantable gastric banding system 205, including a gastric band 210 configured to form a loop around a portion of a stomach 220 of a patient 200 to form a stoma. The gastric band 210 is preferably wrapped around the cardia or esophageal junction of the stomach 220 to restrict the flow of food passing from the upper portions of the stomach 220 to the lower portions of the stomach 220. The restricted flow of food enhances the satiety signals sensed by the patient 200, which desirably reduces food consumption by the patient 200, which aids the patient 200 in losing weight.

Over time, a physician may need to adjust the degree to which the gastric band 210 constricts the stomach 220. As such, the gastric band 210 may include an inflatable portion 215, which comprises an inflatable cuff that wraps around the stomach 220 of the patient 200. The inflatable portion 215 may be filled with fluid and/or gas. The amount of fluid and/or gas in the inflatable portion 215 defines the degree to which the gastric band 210 constricts the stomach 220 (e.g., a greater amount of fluid and/or gas in the inflatable portion 215 will increase the constriction of the stomach 220). A physician may adjust the amount of fluid and/or gas in the inflatable portion 215 via the access port 235.

The access port 235 is preferably fixed subcutaneously within the body of the patient 200, and is preferably fixed to body tissue including the interior muscle wall of the patient 200. The tube 225 carries or conveys fluid to and from the inflatable portion 215 via the access port 235. One end of the tube 225 couples to the access port 235, and the other end of the tube 225 couples to the inflatable portion 215 of the gastric band 210.

A physician inserts a syringe needle 240 into the patient's body to access the access port 235, and varies the amount of fluid in the inflatable portion 215 of the gastric band 210. Generally, the physician must attempt to locate a septum 230 of the access port 235 to pass the syringe needle 240 through the septum 230. The septum 230 must be penetrated by the syringe needle 240 to allow fluid to enter, or be removed from the access port 235. The physician will typically palpate the area around the access port 235 to locate the septum 230.

FIG. 2B illustrates the gastric banding system 205 of FIG. 2A outside the patient's body in an exploded view. As shown, the gastric banding system 205 may comprise the gastric band 210 (comprising the ring 207 and the inflatable portion 215), the tube 225 and the access port 235 (comprising the septum 230). A belt and buckle system for securing the gastric band 210 about a patient's esophageal-gastric junction is omitted for clarity and ease of understanding.

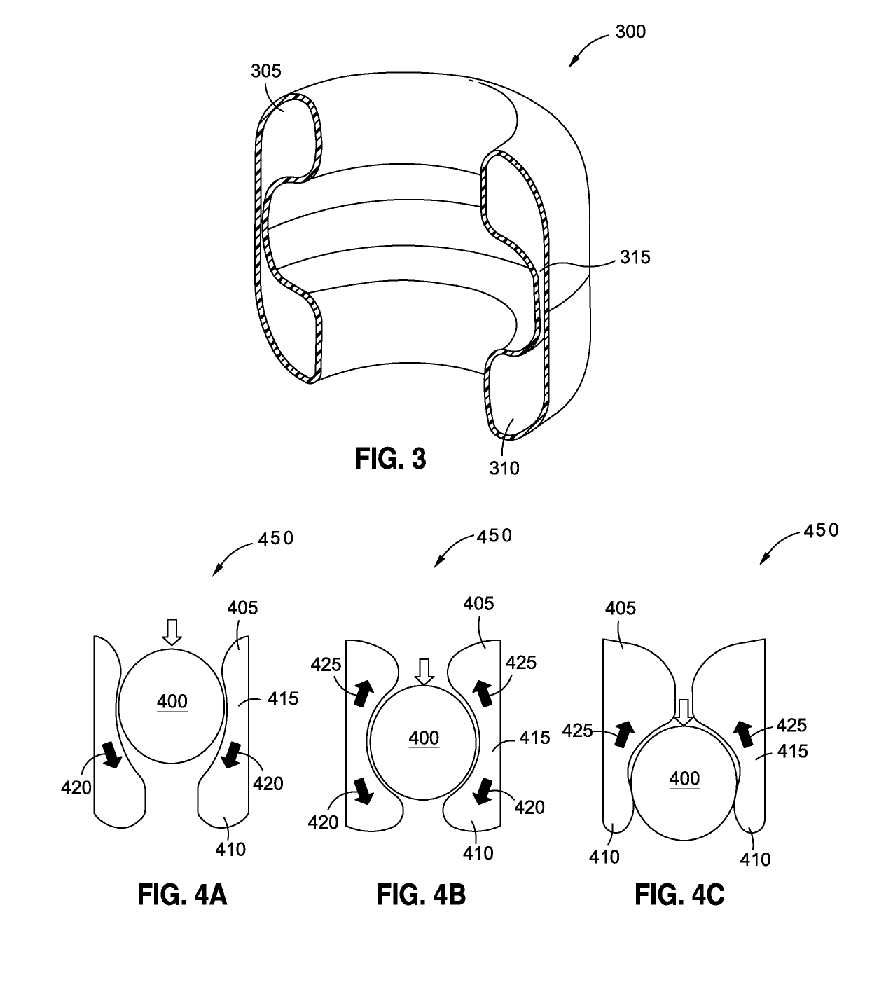

FIG. 3 illustrates one embodiment of a dual-ring reservoir band 300, which may be the inflatable portion 215 of the gastric banding system 205. The dual-ring reservoir band 300 may be rib-shaped and may comprise two or more annular reservoirs or rings for holding fluid (e.g., a top reservoir or ring or tube 305 and a bottom reservoir or ring or tube 310) which can be configured with respect to shape, size or elasticity. The restriction on the patient's esophageal-gastric junction caused by the dual-ring reservoir band 300 (and the flow of fluid into and out of the top reservoir 305 and the bottom reservoir 310) may be determined by the wall thickness and elasticity at different locations of the dual-ring reservoir band 300. In addition, the volume range of the dual-ring reservoir band 300 may be wider than a conventional gastric band, thereby allowing the dual-ring reservoir band 300 to be less sensitive to errors associated with volume fill levels. In turn, the volume range associated with green zone adjustments is widened, making pressure spikes that would normally be associated with the red zone less prevalent. Furthermore, since the dual-ring reservoir band 300 makes more effective use of the green zone (e.g., by better controlling intraluminal pressures associated with the green zone and staying in the green zone for longer durations), the patient may benefit by requiring fewer adjustments.

As shown, the top reservoir 305 and the bottom reservoir 310 may be connected by a fluid transfer section 315. The fluid transfer section 315 may be a wide, virtually 360°, bi-directional fluid transfer section. When implanted into the patient's body, the top reservoir 305 and the bottom reservoir 310 may contact the patient's esophagus, while the fluid transfer section 315 might not contact the patient's esophagus.

As a bolus of food is swallowed by the patient and works its way down to the location of the dual-ring reservoir band 300, the dual-ring reservoir band 300 may self-adjust to allow the bolus of food to pass while maintaining a proper amount of constriction on the patient's esophagus to produce the satiety-increasing effects.

More particularly, FIGS. 4A-4C illustrates the fluid flow within a dual-ring reservoir band 450 (which, for example, may be the dual-ring reservoir band 300) in response to the passage of a bolus of food 400. FIGS. 4A-4C are cross-sectional views. Here, the rib-shape of the dual-ring reservoir band 450 helps peristaltic activity to work more effectively by slowing down the digestion of the food bolus and prolonging peristalsis. Furthermore, a “gating” effect with respect to the fluid transfer between a top reservoir 405 and a bottom reservoir 410 via a fluid transfer section 415 may cause the bolus 400 to be further broken down as a result of the pressures on the esophagus.

Turning to FIG. 4A, the bolus 400 is at a location of a top reservoir 405. The pressure from the presence of the bolus 400 causes fluid located within the top reservoir 405 to move to the fluid transfer section 415 before moving to the bottom reservoir 410, substantially along the direction of arrows 420. Here, the bolus 400 also applies constriction and gates off the bottom reservoir 410 below the bolus 400 thereby slowing the digestion process and helping the patient feel satiated for a longer period of time.

As the bolus 400 moves to the position as shown in FIG. 4B (proximal to the fluid transfer section 415), the fluid within the fluid transfer section 415 is now dispersed to both the top reservoir 405 (e.g., in the direction shown by arrows 425) and the bottom reservoir 410 (e.g., in the direction shown by arrows 420), thereby facilitating the move of the bolus 400 downwards while also applying constriction on the esophagus at the location of both the top reservoir 405 and the bottom reservoir 410.

FIG. 4C shows the bolus 400 proximal to the bottom reservoir 410, having moved past the top reservoir 405. Here, the bolus 400 causes a pressure on the bottom reservoir 410, which in turn, causes the fluid moving to or transferred to the top reservoir 405. As shown, the top reservoir 405 bulges inward due to the influx of fluid thereby resulting in a gating effect. That is, the influx of fluid moving to the top reservoir 405 momentarily prevents any other bolus from passing through the top reservoir 405.

Satiety may be correlated with bolus activity about the gastric band (e.g., moving up and back down), and therefore, in the manner illustrated in FIGS. 4A-4C, the patient may experience improved satiety after swallowing a bolus of food. In addition, the gating effect may assist to guide the bolus 400 through the dual-ring reservoir band 450 by facilitating a funnel shape at the top part of the band, thus reducing the chance for food obstructions leading to potentially adverse events and/or discomfort for the patient. It is further possible that the behavior of the dual-ring reservoir band 450 may help in bolus breakdown and increase bolus activity above the dual-ring reservoir band 450.

Basic functionality and structure of the dual-ring reservoir band 450 having been discussed; now attention will be turned to different embodiments.

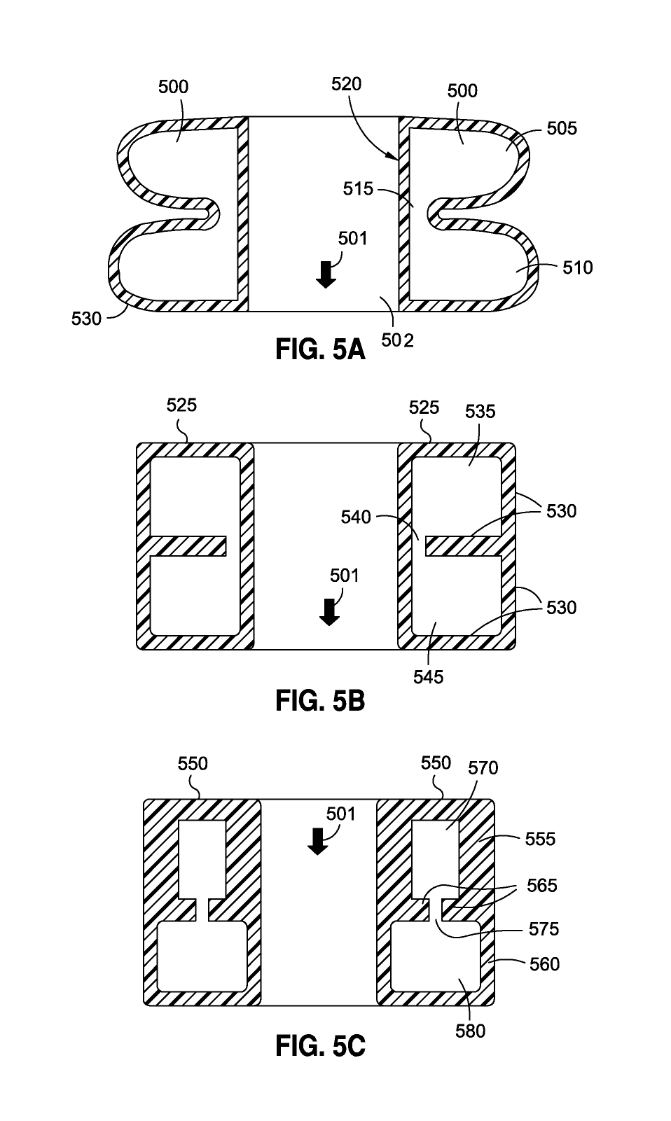

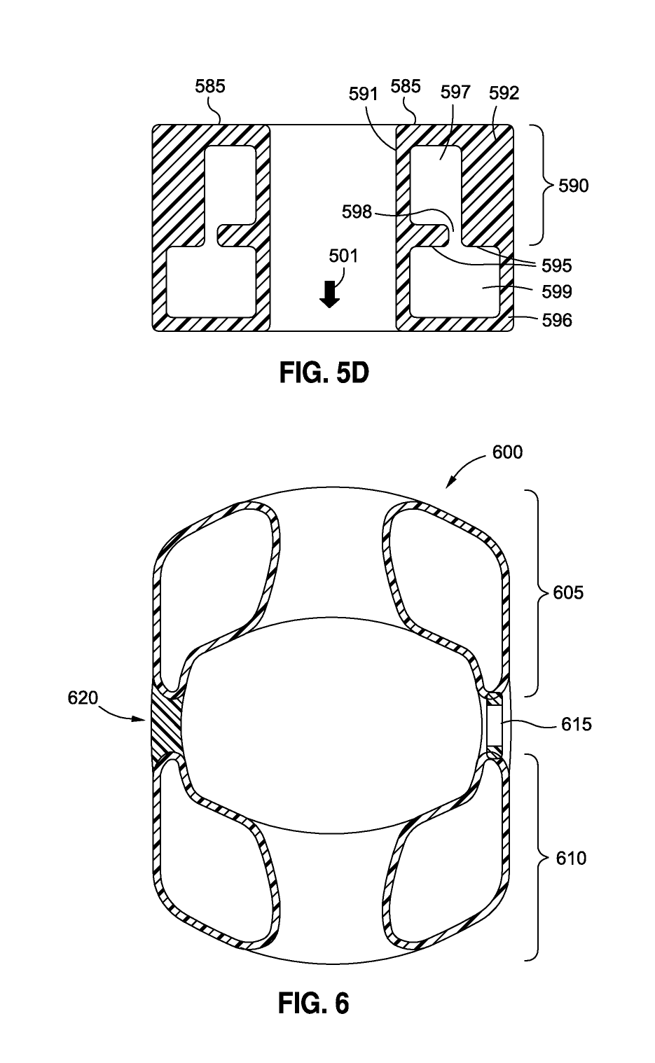

FIG. 5A illustrates a cross sectional view of a rib-shaped, dual-ring reservoir band 500 disposed about an esophagus 502 of a patient oriented in a manner where a bolus of food swallowed by a patient travels down pathway 501 of the esophagus 502 in the direction of arrow 501. The dual-ring reservoir band 500 may include a first reservoir or ring 505 connected to a second reservoir or ring 510 via a fluid transfer section 515. As shown, the dual-ring reservoir band 500 may provide a greater contact area along a substantially smooth inner surface 520, which may lead to increased satiety. Structurally, the first reservoir or ring 505, the second reservoir or ring 510 and the fluid transfer section 515 may be integrated with each other, and may be defined by a compliant wall 530.

FIG. 5B illustrates another embodiment of a dual-ring reservoir band 525. Here, the dual-ring reservoir band 525 may be rectangular when viewing the cross-section of the dual-ring reservoir band 525. One advantage to employing a rectangular shaped (e.g., a square-shaped or box-shaped) dual-ring reservoir band is that the laparoscopic procedure needed to pull the dual-ring reservoir band 525 around the patient's esophagus may be easier and less likely to suffer from errors. In this embodiment, the walls 530 defining a first reservoir or ring 535, a fluid transfer section 540, and a second reservoir or ring 545 may be of uniform thickness, thereby maintaining a consistent fluid transfer rate between the first reservoir or ring 535 and the second reservoir or ring 545 via the fluid transfer section 540.

FIG. 5C illustrates another embodiment of a dual-ring reservoir band 550. Here, the dual-ring reservoir band 550 may have various wall thicknesses in different reservoirs or rings. In this embodiment, the wall 555 defining a first reservoir or ring 570 may be thicker (resulting in a smaller chamber for the first reservoir or ring 570) than the wall 560 defining a second reservoir or ring 580 (which may have a larger reservoir due to the relatively thinner, more elastic or compliant walls). In this manner, the compliance of the dual-ring reservoir band 550 may be varied as a bolus moves downward through the constriction caused by the dual-ring reservoir band 550. In this example, the first reservoir or ring 570 may be more restrictive (due to the thicker walls and increase in fluid transfer timing) than the second reservoir or ring 580 thereby keeping the bolus at the location of the first reservoir or ring 570. Here, the wall 565 corresponding to the fluid transfer section 575 may be even thicker than the walls 555 and 560 to result in a relatively rigid fluid transfer section 575.

FIG. 5D illustrates yet another embodiment of a dual-ring reservoir band 585. Here, the dual-ring reservoir band 585 may have reservoirs having non-uniform wall thicknesses to produce the desired effects. In this embodiment, the wall 590 defining a first reservoir or ring 597 may be unbalanced, having a thinner wall portion 591 proximal to the pathway of the bolus illustrated by arrow 501 and a thicker wall portion 592 distal to the pathway 501. Furthermore, the overall wall thickness of the wall 590 may be thicker (resulting in a smaller chamber in terms of volume for the first reservoir or ring 597) than the wall 596 defining a second reservoir or ring 599 (which may have a larger reservoir due to the relatively thinner, more elastic or compliant walls). In this manner, the compliance of the dual-ring reservoir band 585 may be varied as a bolus moves downward through the constriction caused by the dual-ring reservoir band 585. In this example, the presence of a bolus (and the pressure caused therefrom) may have a higher effect on reservoirs thereby having a more dramatic effect on fluid transfer from one reservoir to another reservoir via the fluid transfer section 598 defined by the wall portions 595.

In another embodiment (not shown), the configuration of the dual-ring reservoir band 585 may be flipped such that the top reservoir might have thinner, uniform walls, while the bottom reservoir might have a thicker, non-uniform wall. In this example, the larger top reservoir would then be more compliant, while the smaller bottom reservoir would then be more restrictive as the bolus travels through.

In yet another embodiment, a dual-ring reservoir band 600 may include a bi-directional valve or two uni-directional valves (in opposite directions) serving as the fluid transfer mechanism. FIG. 6 illustrates an example of a dual-ring reservoir band 600 having a bi-directional valve 615 situated between a first reservoir or ring 605 and a second reservoir or ring 610. As shown, the first reservoir or ring 605 is completely separated from the second reservoir or ring 610. The bi-directional valve 615 may function to regulate flow of fluid between the reservoirs. The bi-directional valve 615 could be adjustable, or contain different configurations for looser or tighter settings, translating into more/less compliance. The bi-directional valve 615 may be configured to be any of a number of different diameters (for the opening). That is, a larger valve diameter increases the flow rate and decreases the amount of constriction on the esophagus during bolus transit, and conversely, a smaller valve diameter decreases the flow rate and increases the amount of constriction on the esophagus during bolus transit. Additionally, or alternatively, the bi-directional valve 615 may be electro-mechanically controlled via inductive means such that the valve diameter may be adjusted either remotely or in response to a bolus to control the degree of constriction on the patient's esophagus. In an embodiment employing the bi-directional valve 615, an access port and tube might not be omitted. However, in some circumstances, the access port and tube may be included for emergency fluid removal and/or initial fluid injection.

As shown in the cross-sectional illustration of FIG. 6, a silicone ring portion 620 may be used to attach the first reservoir or ring 605 and the second reservoir or ring 610. The bi-directional valve 615 may be embedded or housed within the silicone ring portion 620 to fluidly couple the first reservoir or ring 605 to the second reservoir or ring 610.

Certain advantages of a dual-ring system including one or more valves may include increased effective stimulation by altering back and forth between reservoirs contacting the esophagus, more controlled and accurate constriction by electro-mechanical means, and remote adjustments replacing needle/syringe-based adjustments. The valves may be electrical and/or mechanical valves.

One or more funnels can also be implemented into a gastric banding system to induce satiety and/or for guiding a bolus through the gastric band. As a result, improved use of the green zone may be achieved. Furthermore, the funnel may allow for more compliance, which as discussed above, may reduce the number of food obstructions, as well as increase the variety of foods allowed to be eaten by the patient.

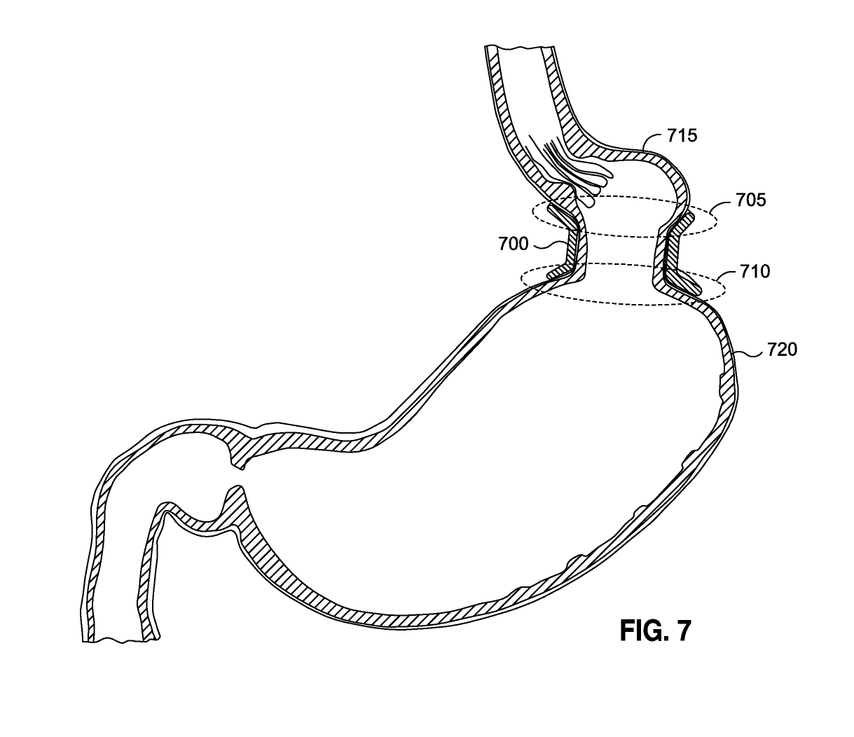

FIG. 7 illustrates an example of a location where a funnel-shaped gastric band 700 may be positioned within a patient's esophageal-gastric junction (which may be the region between an esophagus 715 and a stomach 720). Here, the funnel portion as shown in area 705 provides a streamlined path for the bolus of food and also prevents lateral bulging of the tissue that can impede efficient flow through the narrow constriction. In addition, the shape of the funnel portion also prevents the formation of an inadvertent esophageal dilatation and pouch formation just above the gastric band. This undesired pouch or dilatation can result in dormant/residual food in the pouch which will eventually decay and may even result in a surgical explantation of the gastric band. More particularly, the funnel geometry supports the esophageal tissue just above the gastric band and prevents the formation of the pouch.

In addition, the inverted funnel geometry at the other end of the gastric band in area 710, which faces the stomach 720 provides a more conformal fit with the geometry of the larger stomach and can prevent slippage of the gastric band by acting as a positional anchor. In addition, the prevention of erosion is also attained by the inverted funnel geometry. In this manner, the v-shaped funnel and the inverted v-shaped funnel provide many advantages to the patient.

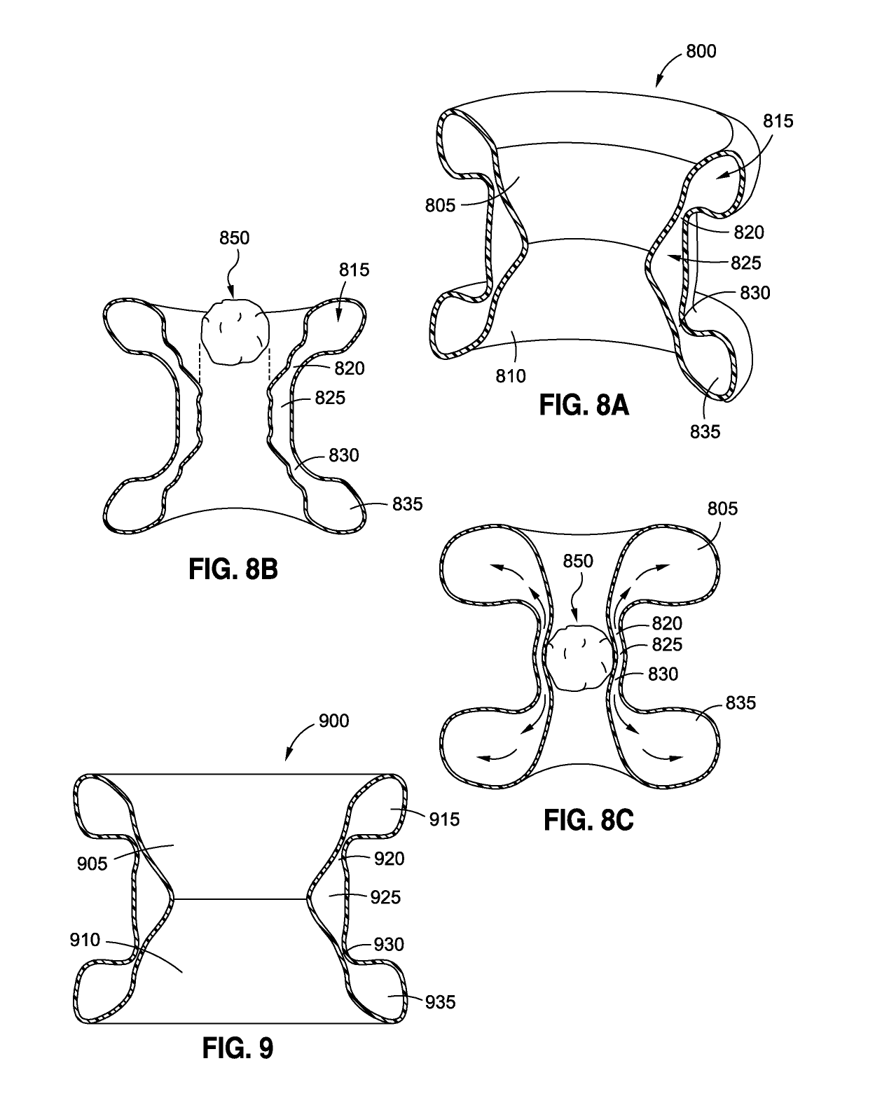

In one embodiment, one or more funnel-like mechanisms may be implemented into a dual-ring system 800. For example, FIG. 8A illustrates an embodiment of a dual-funnel, dual-ring (DFDR) system 800. The DFDR system 800 may include a first funnel portion 805 fluidly coupled to a second funnel portion 810. The DFDR system 800 may be implanted in the patient to constrict a lower esophagus or upper stomach region in a manner allowing a swallowed bolus to reach the first funnel portion 805 before reaching the second funnel portion 810. As such, the first funnel portion 805 is a “V” shaped funnel, thereby guiding the bolus to the portion of the DFDR system 800 which corresponds with the middle pouch area 825.

The first funnel portion 805 may also include a top reservoir or ring 815. The second funnel portion 810 may include a bottom reservoir or ring 835. In the area where the first funnel portion 805 and the second funnel portion 810 meets, another fluid reservoir (a middle pouch 825) may be positioned. Fluid transfer sections 820 and 830 may be included to facilitate the transfer of fluid among the top reservoir or ring 815, the middle pouch 825 and the bottom reservoir or ring 835.

As shown in FIGS. 8B and 8C, the first funnel portion 805 guides a bolus 850 to an area proximal to the middle pouch 825, and when the bolus 850 contacts the middle pouch 825, it applies pressure to the middle pouch 825, thereby causing a transfer of fluid from the middle pouch 825 to the top reservoir or ring 815 and the bottom reservoir or ring 835 via fluid transfer sections 820 and 830, respectively. In other words, the middle pouch 825 is the only high-pressure contact area, which displaces the fluid to either the top reservoir or ring 815 or the bottom reservoir or ring 835. Here, at this stage, the top reservoir or ring 815 and the bottom reservoir or ring 835 expand in response to receiving the fluid, and function substantially as fluid reservoirs and do not contact the bolus 850 which has now moved to the location proximal to the middle pouch 825. The transfer of fluid out of the middle pouch 825 widens the opening at the middle pouch 825, and the bolus 850 may move past the location of the middle pouch 825 to the second funnel portion 810.

Referring back to FIG. 8A, the second funnel portion 810 is an inverted “V” shape, and allows the fluid from the top reservoir or ring 815 and the bottom reservoir or ring 835 to move back to the middle pouch 825 via fluid transfer sections 820 and 830, respectively, once the bolus 850 is no longer applying pressure to the middle pouch 825.

Variations to the DFDR system 800 may include altering the size of the fluid transfer sections, adding one or more valves (in place of and/or in addition to the fluid transfer sections), or essentially removing the fluid transfer section 830 and the bottom reservoir or ring 835, among other modifications.

For example, FIG. 9 illustrates an embodiment of a dual-funnel, dual-ring (DFDR) system 900. Similar in structure and function to the DFDR system 800 of FIG. 8A, the DFDR system 900 of FIG. 9 may include a “V” shaped first funnel portion 905 and an inverted “V” shaped second funnel portion 910, a top reservoir or ring 915 fluidly connected to a first fluid transfer section 920, which in turn is fluidly connected to a first side of a middle pouch 925. The middle pouch 925 may be configured to contact a bolus (e.g., when the bolus is larger than a predetermined size) as it travels through the constriction formed by the middle pouch 925. In addition, the middle pouch 925 may be fluidly connected on a second side to a second fluid transfer section 930, which in turn is fluidly coupled to a bottom reservoir or ring 935.

One difference between the DFDR system of FIG. 9 and the DFDR system of FIG. 8 is that the first and second fluid transfer sections 920 and 930 are configured to be smaller than the corresponding fluid transfer sections 820 and 830 of FIG. 8A. The reduced size of the fluid transfer sections 920 and 930 may cause a longer fluid transfer time when a bolus is contacting the middle pouch 925. One advantage of such a configuration is that the patient may realize longer periods of satiety.

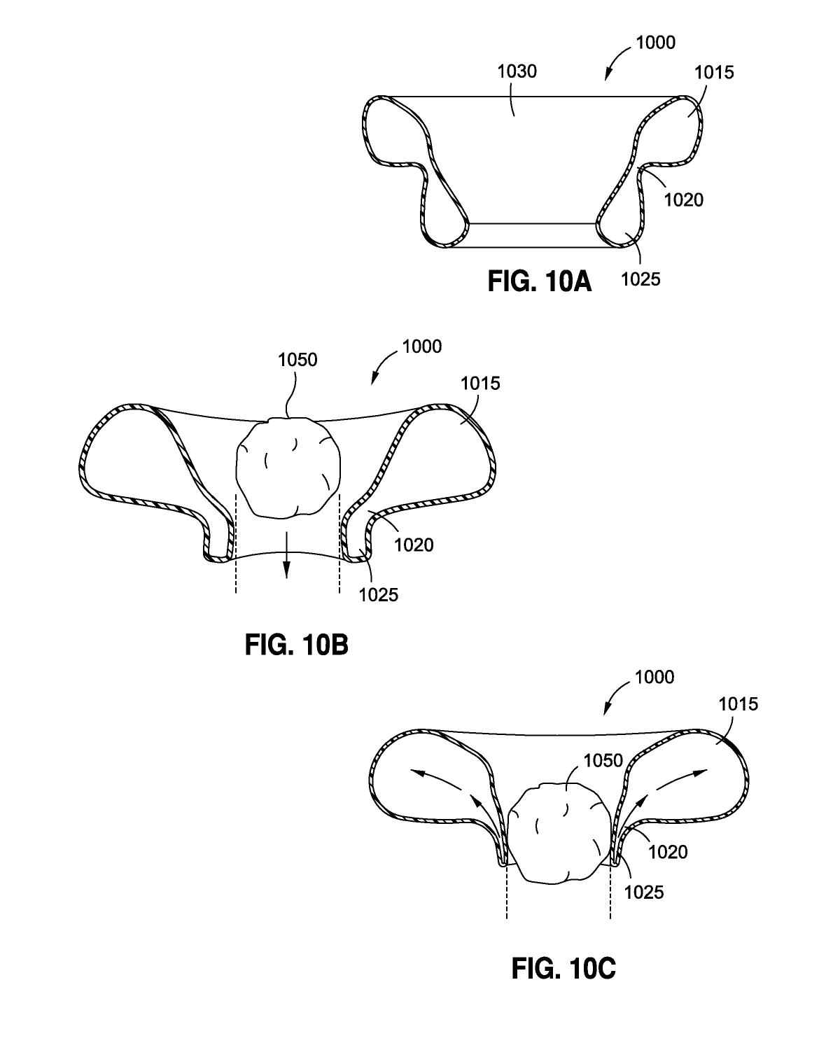

As mentioned, another embodiment may result from essentially removing the fluid transfer section 830 and the bottom reservoir or ring 835 of FIG. 8A. Such an embodiment is illustrated in FIGS. 10A-10C.

As shown in FIG. 10A, the single-ring, single funnel (SRSF) system 1000 may include a reservoir or ring 1015 fluidly connected to a fluid transfer section 1020, which in turn is fluidly connected to a pouch or second reservoir 1025. Here, the SRSF system 1000 defines an inwardly-sloped or funneling surface 1030 which creates a gradually decreasingly-sized opening for a bolus 1050 moving from the first reservoir or ring 1015 down to the pouch or second reservoir 1025. In this manner, the sloped outer surface 1030 is able to guide the bolus 1050 to the pouch or second reservoir 1025.

As further illustrated in FIGS. 10B and 10C, when the bolus 1050 contacts the pouch or second reservoir 1025, it applies pressure to the pouch or second reservoir 1025, thereby causing a transfer of fluid from the pouch or second reservoir 1025 to the reservoir or ring 1015 via fluid transfer section 1020. Here, at this stage, the top reservoir or ring 1015 expands in response to receiving the fluid. The transfer of fluid out of the pouch or second reservoir 1025 widens the opening at the pouch or second reservoir 1025, and the bolus 1050 may move past the location of the pouch or second reservoir 1025.

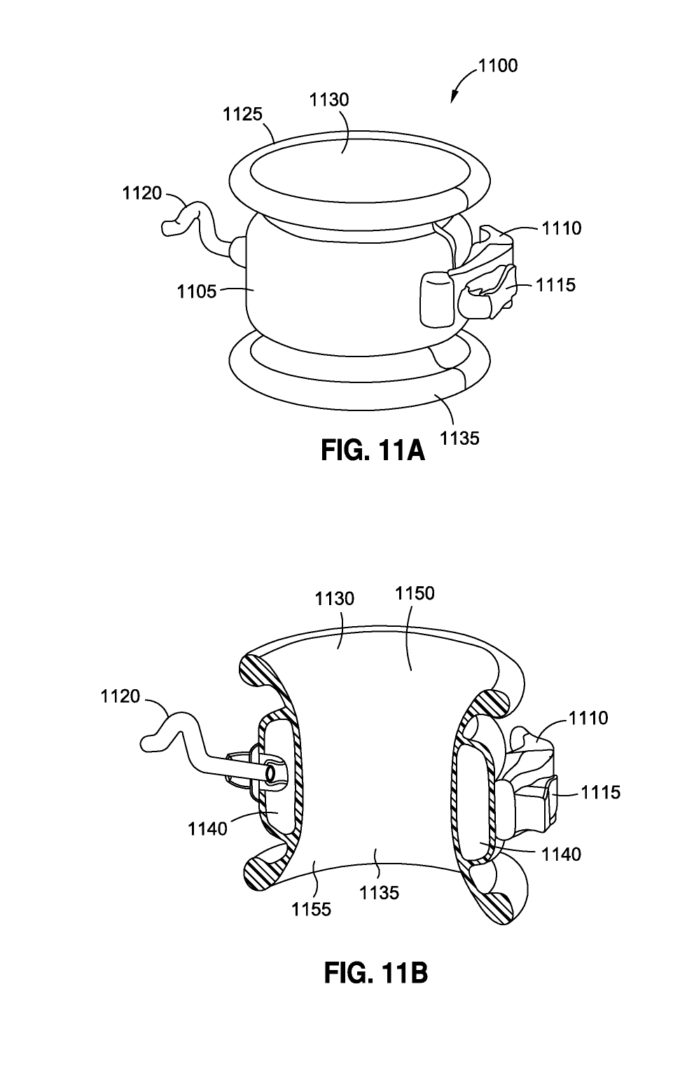

FIGS. 11A and 11B illustrate a dual-funnel, single-band, (DFSB) system 1100. The DFSB system 1100, like the other systems described herein (e.g., systems 800, 900, 1000) may be a gastric banding system for placement around an exterior of a patient's esophageal-gastric junction. The DFSB system 1100 may include a ring 1105 surrounding an inflatable portion or reservoir 1140 coupled to a tube 1120. As with other gastric banding systems, the tube 1120 may be coupled to an access port (not shown). The DFSB system 1100 may further include a belt 1110 for receiving a buckle 1115 to form a substantially circular and smooth contour about a patient's esophageal-gastric junction and for holding the DFSB system 1100 in place when the DFSB system 1100 is implanted. For a smooth transition, the DFSB system 1100 may integrate dual-funnels 1125 on the inside surface of the inflatable portion or reservoir 1140. The dual-funnels 1125 may include a top funnel portion 1130 and a bottom funnel portion 1135 which extend beyond the width of the ring 1105. As shown in FIG. 11B, the top funnel portion 1130 may include an inwardly sloped or funnel surface 1150 which may be substantially smooth. The inwardly sloped or funneled surface 1150 may be formed of a silicone rubber or other biocompatible material, and may function to guide a bolus through the DFSB system, thereby allowing for inadvertent larger boluses and possibly decreasing the number of obstructions.

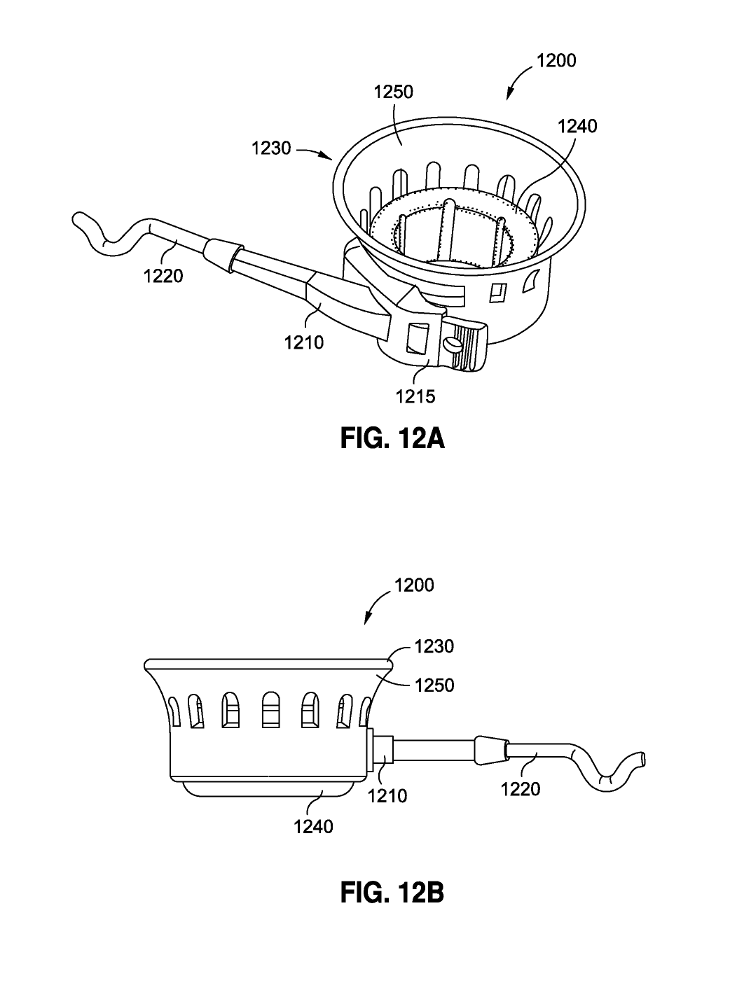

FIGS. 12A and 12B illustrate a single-funnel, single-band, (SFSB) system 1200. The DFSB system 1200, like the other systems described herein (e.g., system 800, 900, 1000) may be a gastric banding system for placement around an exterior of a patient's esophageal-gastric junction. The SFSB system 1200 may include a ring 1205 surrounding an inflatable portion or reservoir 1240 coupled to a tube 1220. As with other gastric banding systems, the tube 1220 may be coupled to an access port (not shown). The SFSB system 1200 may further include a belt 1210 for receiving a buckle 1215 to form a substantially circular and smooth contour about a patient's esophageal-gastric junction and for holding the SFSB system 1200 in place when the SFSB system 1200 is implanted. Unlike traditional gastric banding systems, the SFSB system 1200 may integrate a single funnel 1225 on the outside surface of the inflatable portion or reservoir 1240. As shown in FIG. 12B, the funnel portion 1230 may include an inwardly sloped or funnel surface 1250 which may be substantially smooth. Here, the funnel portion 1230 also includes optional slits or holes. The row of slits or holes may provide effective tissue support while still increasing compliance of the SBSB system 1200. That is, the row of slits or holes does not impede contiguous tissue support. The inwardly sloped or funneled surface 1250 may be formed of a silicone rubber or other biocompatible material, and may function to guide a bolus through the SFSB system, thereby allowing for inadvertent larger boluses and possibly decreasing the number of obstructions. By eliminating a bottom funnel portion (as compared to the DFSB system 1100 of FIG. 11), cost savings may be achieved by manufacturing a gastric banding system with only one funnel.

Certain embodiments have been disclosed to clarify the concepts including the above structural configurations. However, one skilled in the art will recognize that an endless number of implementations may be performed with the concepts herein. For example, the tube may be a catheter and may be used in other applications which require transferring fluid or gas.

Unless otherwise indicated, all numbers expressing quantities of ingredients, volumes of fluids, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An inflatable apparatus for use within a gastric banding system for the treatment of obesity, the inflatable apparatus comprising:
a first fluid reservoir having a first inner surface configured to contact and form a constriction about a first portion of a patient's stomach;
a second fluid reservoir having a second inner surface configured to contact and form a constriction about a second portion of the patient's stomach; and
a bi-directional fluid transfer component positioned between the first fluid reservoir and the second fluid reservoir for improving the compliance of the first fluid reservoir and the second fluid reservoir, the bi-directional fluid transfer component configured to transfer fluid from the first fluid reservoir to the second fluid reservoir in response to a bolus exerting a pressure on the first inner surface of the first fluid reservoir, and further configured to transfer fluid from the second fluid reservoir to the first fluid reservoir in response to the bolus exerting a pressure on the second inner surface of the second fluid reservoir.

2. The inflatable apparatus of claim 1, wherein the bi-directional fluid transfer component is further configured to transfer fluid from the first fluid reservoir to the second fluid reservoir in response to the removal of the pressure exerted by the bolus on the second inner surface of the second fluid reservoir.

3. The inflatable apparatus of claim 2, wherein the bi-directional fluid transfer component is further configured to transfer fluid from the second fluid reservoir to the first fluid reservoir in response to the removal of the pressure exerted by the bolus on the first inner surface of the first fluid reservoir.

4. The inflatable apparatus of claim 1, wherein the first fluid reservoir and the second fluid reservoir are rib-shaped, and further wherein the first inner surface and the second inner surface are substantially smooth to provide a contact area for contacting the patient's stomach.

5. The inflatable apparatus of claim 1, wherein the first fluid reservoir is defined by a first set of walls, and the second fluid reservoir is defined by a second set of walls, further wherein the first set of walls and the second set of walls are of rectangular cross section and uniform in thickness.

6. The inflatable apparatus of claim 1, wherein the first fluid reservoir is defined by a first set of walls of a first thickness, and the second fluid reservoir is defined by a second set of walls of a second thickness greater than the first thickness, further wherein the first set of walls and the second set of walls are each of rectangular cross section.

7. The inflatable apparatus of claim 1, wherein the first fluid reservoir is defined by a first set of walls of a first thickness, and the second fluid reservoir is defined by a second set of walls of a second thickness less than the first thickness, further wherein the first set of walls and the second set of walls are each of rectangular cross section.

8. The inflatable apparatus of claim 1, wherein the first fluid reservoir is defined by a first set of walls having non-uniform thickness, and the second adjustably filled fluid reservoir is defined by a second set of walls having uniform thickness.

9. The inflatable apparatus of claim 1, wherein the first fluid reservoir is defined by a first set of walls having uniform thickness, and the second fluid reservoir is defined by a second set of walls having non-uniform thickness.

10. A gastric banding device comprising an inflatable portion for the treatment of obesity, the inflatable portion comprising:
a first fluid reservoir configured to displace fluid within the first fluid reservoir in response to a bolus causing pressure on the first fluid reservoir;
a second fluid reservoir separated from the first fluid reservoir, and configured to receive the displaced fluid from within the first fluid reservoir, and further configured to displace fluid from within the second fluid reservoir in response to the bolus causing pressure on the second fluid reservoir; and
a first valve for allowing fluid communication between the first fluid reservoir and the second fluid reservoir.

11. The gastric banding device of claim 10, wherein the first valve is a bi-directional valve positioned between the first fluid reservoir and the second fluid reservoir, and configured to transfer the displaced fluid from within the first fluid reservoir to the second fluid reservoir, and further configured to transfer the displaced fluid from within the second fluid reservoir to the first fluid reservoir.

12. The gastric banding device of claim 10, wherein the inflatable portion further comprises a second valve, and wherein the first valve is a uni-directional valve positioned between the first fluid reservoir and the second fluid reservoir for transferring the displaced fluid from within the first fluid reservoir to the second fluid reservoir, and further wherein the second valve is a uni-directional valve positioned between the first fluid reservoir and the second fluid reservoir for transferring the displaced fluid from within the second fluid reservoir to the first fluid reservoir.

13. The gastric banding device of claim 11, wherein the first valve is electromechanically controlled.

14. The gastric banding device of claim 11, wherein the first valve is induction controlled.

15. A dual-reservoir, dual-funnel gastric banding device usable for the treatment of obesity, comprising:
a first fluid reservoir forming a top section of a funnel portion;
a second fluid reservoir forming a bottom section of an inverted funnel portion;
a middle pouch forming a bottom section of the funnel portion, and a top section of the inverted funnel portion;
a first fluid transfer component forming a middle section of the funnel portion between the first fluid reservoir and the middle pouch for transferring fluid between the first fluid reservoir and the middle pouch; and
a second fluid transfer component forming a middle section of the inverted funnel portion between the second fluid reservoir and the middle pouch for transferring fluid between the second fluid reservoir and the middle pouch.

16. The dual-reservoir, dual-funnel gastric banding device of claim 15, wherein the funnel portion is configured to guide a bolus to an area proximal to the middle pouch.

17. The dual-reservoir, dual-funnel gastric banding device of claim 15, wherein when a bolus contacts the middle pouch and exerts a pressure thereupon, some fluid within the middle pouch is transferred to the first reservoir via the first fluid transfer component and some fluid within the middle pouch is transferred to the second reservoir via the second fluid transfer component to allow the bolus to pass through the middle pouch.

18. A single-reservoir, single-funnel gastric banding device usable for the treatment of obesity, comprising:
a first fluid reservoir forming a top section of a funnel portion configured to guide a bolus swallowed by a patient;
a second fluid reservoir forming a bottom section of the funnel portion; and
a fluid transfer component forming a middle section of the funnel portion, and for transferring fluid between the first reservoir and the second reservoir.

19. The single-reservoir, single-funnel gastric banding device of claim 18, wherein when a bolus contacts the second fluid reservoir and exerts a pressure thereupon, some fluid within the second fluid reservoir is transferred to the first fluid reservoir via the fluid transfer component to allow the bolus to pass through.

20. A gastric banding system for the treatment of obesity, the gastric banding system comprising:
an inflatable portion configured to provide constriction on a stomach of a patient;
a ring coupled to an outside surface of the inflatable portion configured to provided structural support;
an funnel portion integrated with an inside surface of the inflatable portion, the funnel portion configured to guide a bolus swallowed by the patient;
an access port for the addition of fluid to and removal of fluid from the inflatable portion; and
a tube for fluidly connecting the inflatable portion and the access port.

21. The gastric banding system of claim 20, wherein the funnel portion includes a slot.

22. The gastric banding system of claim 20, wherein the funnel portion includes a v-shaped section and an inverted v-shaped section.
